(19) United States
(12) Patent Application Publication
(10) Pub. No.: US 2012/0135488 A1
(43) Pub. Date: May 31, 2012

(54) METHODS FOR THE PREPARATION AND USE OF CELLULOSIC FEEDSTOCK FOR ETHANOL PRODUCTION

(75) Inventor: James B. Garrett, San Diego, CA (US)

(73) Assignee: BP Corporation North America, Inc., Naperville, IL (US)

(21) Appl. No.: 13/319,232

(22) PCT Filed: Jun. 22, 2010
(86) PCT No.: PCT/US2010/039460

§ 371 Date: Feb. 16, 2012

Related U.S. Application Data
(60) Provisional application No. 61/219,362, filed on Jun. 22, 2009.

(51) Int. Cl. C12P 7/10; C08B 30/00

(52) U.S. Cl. 435/165; 127/34

(57) ABSTRACT

The instant invention provides methods for increasing the efficiency and yield of cellulosic ethanol production.

### RELATED APPLICATIONS

This applications claims the benefit of U.S. Provisional Application No. 61/219,362, filed 22 Jun. 2009, the entire contents of which are expressly incorporated herein by reference.

### BACKGROUND OF THE INVENTION

Cellulosic ethanol is a bio fuel produced from wood, grasses, or the non-edible parts of plants. Cellulosic ethanol is produced from lignocellulose, a structural material that comprises much of the mass of plants. Lignocellulose is composed mainly of cellulose, hemicellulose and lignin. Corn stover, switchgrass, miscanthus, woodchips and the byproducts of lawn and tree maintenance are some of the more commonly known cellulosic materials for ethanol production. Production of ethanol from lignocellulose has the advantage of abundant and diverse raw material compared to sources like corn and cane sugars, but requires a greater amount of processing to make the sugar monomers available to the microorganisms that are typically used to produce ethanol by fermentation.

For commercial scale production of cellulosic ethanol, it is important that the process of producing ethanol from lignocellulose is as efficient as possible. Accordingly, new and improved methods for the preparation and use of feedstock are necessary to make cellulosic ethanol production a commercially viable method of ethanol production.

### SUMMARY OF THE INVENTION

The instant invention is based, at least in part, on the surprising discovery that the juice produced by the initial milling of feedstock used in the production of cellosic ethanol contains sugar. Initially, it was believed that this juice did not contain enough, or any, sugar, and therefore methods for re-integrating the juice downstream of the hydrolyzer were not necessary.

However, the discovery by the inventors that this juice contained measurable and significant levels of sugar motivated the inventors to design a process whereby the juice could be reintroduced to the ethanol production process in order to increase the amount of ethanol produced and the efficiency of the production process. For example, in certain embodiments, the invention provides processes for preparation, e.g., juicing, of cane prior to further treatment. The processes advantageously reduce the formation of the fermentation inhibitor 5-HFM.

Accordingly, in one aspect, the invention provides methods for preparing feedstock comprising, milling the feedstock and separating the juice and the bagasse; thereby preparing the feedstock.

In one embodiment, the methods of the invention further comprise allowing microbes to consume the residual soluble sugar in the bagasse after separating the juice.

In another related embodiment, the microbes are present on the feedstock at harvest. In an alternate embodiment, the feedstock is inoculated with the microbes after the juice is extracted.

In a related embodiment, the microbes are allowed to consume the residual sugar for 1 day, 2 days, 3 days, 4 days, 5 days, 6 days, 7 days, 8 days, 9 days, 10 days, 11 days, 12 days, 13 days or 14 days. In another embodiment, the feedstock is fermented to produce ethanol.

In another embodiment, the juice is added back to the bagasse mixture prior to fermentation. In yet another embodiment, the is hydrolyzed prior to addition of the juice.

In another embodiment, the processed feedstock is fermented as depicted in FIG. 1.

In another aspect, the instant invention provides methods of increasing the amount of ethanol produced from feedstock by processing the feedstock as depicted in FIG. 1.

In one embodiment, the feedstock is sugarcane.

In another aspect, the instant invention provides methods of increasing the efficiency of ethanol production by fermentation of processed feedstock by a microorganism comprising, milling the feedstock and collecting the juice, wherein the juice comprises sugar, using the juice during the feedstock preparation or fermentation; thereby increasing the efficiency of ethanol production, or the amount of ethanol produced per amount of feedstock. In a related embodiment, the efficiency is increased as compared to a method in which the juice is not used.

In one embodiment, the juice is added to the fermentation broth. In a related embodiment, the fermentation broth is C5 sugar fermentation broth. In another embodiment, the fermentation broth is C6 sugar fermentation broth.

In another embodiment, the juice is used as a wash for a solid/liquid separation step, e.g., a screw press.

In another embodiment, the juice is fermented separately from the bagasse extract.

In another embodiment, the juice is used to grow yeast to produce yeast extract. In a related embodiment, the yeast is used to produce yeast extract.

In another embodiment, the juice is used to grow microorganisms that produce cellulosic enzymes. In a related embodiment, the cellulosic enzymes are used in the preparation of C6 sugar extract.

In another embodiment, the juice is used to culture bacteria or yeast for fermentation of C5 or C6 sugar extract.

In another embodiment, the feedstock is energy cane, e.g., sugar cane.

### DESCRIPTION OF THE DRAWINGS

FIG. 1 depicts a schematic view of the ethanol production process from milling of the feedstock to the fermentation of the C5 and C6 sugars as well as downstream uses of the juice.

### DETAILED DESCRIPTION OF THE INVENTION

Production of ethanol from biomass is a viable approach to produce fuel grade ethanol. In order to make the production of ethanol from biomass commercially viable the process needs to become more efficient from the growth and harvest of the feedstock to purification of the ethanol.

Currently, feedstock is harvested and prepared for storage by milling the material to remove as much of the water (called “juice” in the industry) content as possible. Once the juice is removed the feedstock can be stored for extended periods of time until it is ready for further processing and fermentation. For example, feedstock that has had the juice removed can be stored for 1, 2, 3, 4, 5, 6, 7, 8, 9, 10, 11, 12, 13, or 14 days or more or 2, 3, 4, 5, 6, 12, or 18 months or more.

Stored biomass is hydrolyzed and separated to yield solutions containing C5 and C6 sugars that are fermented to produce ethanol. Until the time of the instant invention, the juice was thought not to contain any, or enough, sugar to make it worth collecting and re-integrating into the ethanol production method.

As used herein, the term “juice” is intended to mean the water-based liquid that is extracted from feedstock upon milling, i.e., with a roller mill. The juice contains 5, 6, 7, 8, 9, 10, 11, 12, 13, 14, 15% or more sugar.

As used herein, the term “bagasse” is intended to mean the fibrous residue remaining after any of the various feedstock stalks are crushed and their juice extracted.

As used herein, “C6 sugar” is intended to mean sugars that have a six carbon backbone. For example, glucose is a common six carbon sugar. Generally, six-carbon sugars can be fermented using conventional yeasts.

As used herein, “C5 sugar” is intended to mean sugars that have a five carbon backbone. For example, C5 sugars are sugars such as xylose. Generally, recombinant bacteria can be used to ferment C5 sugars as common yeasts cannot efficiently ferment C5 sugars to ethanol.

It is understood by those of skill in the art that terms and methods as used herein have equivalent methods that can be substituted for those described herein while still obtaining the same results. For example, methods for milling feedstock, e.g., roller milling feedstock, are described. Additionally, methods of screw pressing feedstock are described. One of ordinary skill in the art understands that these methods can be used interchangeably to obtain the same functional result.

In one embodiment, the sugar in the juice is fermented to ethanol. Fermentation may be carried out by yeast, bacteria or other microbes capable of fermenting the product stream to a desired efficiency and yield. In an embodiment, the fermentation is carried out using genetically engineered yeast or bacteria, for example, but not limited to, Zymomonas or E. coli capable of fermenting the pentose sugars xylose, arabinose, or a combination thereof, in addition to the hexose sugars: glucose, mannose, galactose, or a combination thereof. Those skilled in the art are familiar with the requirements for fermentation of sugar to produce ethanol.

Exemplary feedstocks that can be used in the methods of the invention include miscanthus, e.g., Miscanthus floridulus, Miscanthus giganteus, Miscanthus sacchariflorus, Miscanthus sinensis, Miscanthus tinctorius, Miscanthus transmorrisonensis, Erianthus, e.g., E. acutecarinatus, E. acutipennis-E. adpressus, E. alopecuroides, E. angulatus, E. angustifolius, E. armatus, E. articulatus, E. arundinaceus, E. aspen, E. aureus, E. bakeri, E. balansae, E. beccarii, E. bengalensis, E. biaristatus, E. bifidus, E. birmanicus, E. bolivari, E. brasilianus, E. brevibarbis, E. capensis, E. chrysothrix, E. ciliaris, E. clandestinus, E. coarctatus, E. compactus, E. contortus, E. cumingii, E. cuspidatus, E. decus-sylvae, E. deflorata, E. divaricatus, E. dohrni, E. ecklonii, E. elegans, E. elephantinus, E. erectus, E. fallax, E. fastigiatus, E. filifolius, E. fischerianus, E. flavescens, E. flavipes, E. flavoinflatus, E. floridulus, E. formosanus, E. formosus, E. fruhstorferi, E. fulvus, E. giganteus, E. glabrinodis, E. glaucus, E. griffithii, E. guttatus, E. hexastachyus, E. hookeri, E. hostii, E. humbertianus, E. inhamatus, E. irritans, E. jacquemontii, E. jamaicensis, E. japonicus, E. junceus, E. kajkaiensis, E. kanashiroi, E. lancangensis, E. laxus, E. longesetosus, E. longifolius, E. longisetosus, E. longisetus, E. lugubris, E. luzonicus, E. mackinlayi, E. macratherus, E. malcolmi, E. manueli, E. maximus, E. mishmeensis, E. mollis, E. monstierii, E. munga, E. munja, E. nepalensis, E. nipponensis, E. nudipes, E. obtusus, E. orientalis, E. pallens, E. parviflorus, E. pedicellaris, E. perrieri, E. pictus, E. pollinioides, E. procerus, E. pungens, E. purpurascens, E. purpureus, E. pyramidalis, E. ravennae, E. rehni, E. repens, E. rockii, E. roxburghii, E. rufipilus, E. rufus, E. saccharoides, E. sara, E. scriptorius, E. sesquimetralis, E. sikkimensis, E. smallii, E. sorghum, E. speciosus, E. strictus, E. sukhothaiensis, E. sumatranus, E. teretifolius, E. tinctorius, E. tonkinensis, E. tracyi, E. trichophyllus, E. trinii, E. tristachyus, E. velutinus, E. versicolor, E. viguieri, E. villosus, E. violaceus, E. vitalisi, E. vulpinus, E. wardii, E. williamsii; energy cane, such as sugar cane, e.g., S. acinaciforme, S. aegyptiacum, S. alopecuroides, S. alopecuroideum, S. alopecuroidum, S. alopecurus, S. angustifolium, S. antillarum, S. appressum, S. arenicola, S. argenteum, S. arundinaceum, S. asperum, S. atrorubens, S. aureum, S. balansae, S. baldwini, S. baldwinii, S. barberi, S. barbicostatum, S. beccarii, S. bengalense, S. benghalense, S. bicorne, S. biflorum, S. boga, S. brachypogon, S. bracteatum, S. brasilianum, S. brevibarbe, S. brevifolium, S. brunneum, S. caducum, S. caffrosum, S. canaliculatum, S. capense, S. casi, S. caudatum, S. cayennense, S. chinense, S. ciliare, S. coarctatum, S. confertum, S. conjugatum, S. contortum, S. contractum, S. cotuliferum, S. cylindricum, S. deciduum, S. densum, S. diandrum, S. dissitiflorum, S. distichophyllum, S. dubium, S. ecklonii, S. edule, S. elegans, S. elephantinum, S. erianthoides, S. europaeum, S. exaltatum, S. fallax, S. fasciculatum, S. fastigiatum, S. fatuum, S. filifolium, S. filiforme, S. floridulum, S. formosanum, S. fragile, S. fulvum, S. fuscum, S. giganteum, S. glabrum, S. glaga, S. glaucum, S. glaza, S. grandiflorum, S. griffithii, S. hildebrandtii, S. hirsutum, S. holcoides, S. hookeri, S. hybrid, S. hybridum, S. indum, S. infirmum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetum, S. iota, S. luzonicum, S. macilentum, S. macrantherum, S. maximum, S. mexicanum, S. modhara, S. modhua, S. monandrum, S. moonja, S. munja, S. munroanum, S. muticum, S. narenga, S. nareya, S. negrosense, S. obscurum, S. occidentale, S. officinale, S. officinalis, S. officinarum, S. palisoti, S. pallidum, S. paniceum, S. panicosum, S. pappiferum, S. parviflorum, S. pedicellare, S. perrieri, S. polydactylum, S. polystachyon, S. polystachyum, S. porphyrocomum, S. praegrande, S. procerum, S. propinquum, S. punctatum, S. purpuratum, S. rara, S. rarum, S. ravennae, S. repens, S. reptans, S. revennae, S. ridleyi, S. robustum, S. roseum, S. rubicundum, S. rufipilum, S. rufum, S. sagittatum, S. sanguineum, S. sape, S. sara, S. sarpata, S. scindicus, S. semidecumbens, S. seriferum, S. sibiricum, S. sikkimense, S. sinense, S. sisca, S. soltwedeli, S. sorghum, S. speciosissimum, S. sphacelatum, S. spicatum, S. spontaneum, S. spontaneum, S. stenophyllum, S. stewartii, S. strictum, S. teneriffae, S. tenuius, S. ternatum, S. thunbergii, S. tinctorium, S. tridentatum, S. trinii, S. tripsacoides, S. tristachyum, S. velutinum, S. versicolor, S. viguieri, S. villosum, S. violaceum, S. wardii, S. warmingianum, S. williamsii; hybrids, e.g. L 99-233, L 99-226, L79-1001, L 79-1002, L 99-233, L 99-226, HoCP 91-552, HoCP 91-555, Ho 00-961, Ho 02-113, Ho 03-19, Ho 03-48, Ho 99-51, Ho 99-58, US 72-114, Ho 02-144, Ho 06-9002; sorghum, e.g., Sorghum almum, Sorghum amplum, Sorghum angustum, Sorghum arundinaceum, Sorghum bicolor, Sorghum bicolor subsp. drummondii-Sudan grass, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, Sorghum vulgare, hybrids, e.g., sugar cane x Miscanthus or sugar cane x Erianthus; Napier grass (elephant grass), e.g., Pennisetum purpureum; or switch grass, e.g., Panicum virgatum.

However, as described in the example below, the inventors of the instant invention discovered that the juices contain significant amounts of sugar that can be reintroduced at one or more points in the ethanol production process to increase the efficiency and production of ethanol from feedstock.

Feedstock contains appreciable levels of soluble sugars that hydrolyze under heat and acid to form fermentation inhibitors. For example, sucrose yields glucose and fructose, and fructose forms 5-HMF. 5-HMF is a fermentation inhibitor if it passes through the hydrolyzer into the fermentation media.

Accordingly, in one aspect, the instant invention provides methods for the reduction of 5-HFM.

In one embodiment, the instant invention provides methods for dewatering feedstock, e.g., sugarcane, in order to preserve the feedstock for a longer period of time and in order to increase the amount of ethanol that can be produced from the feedstock.

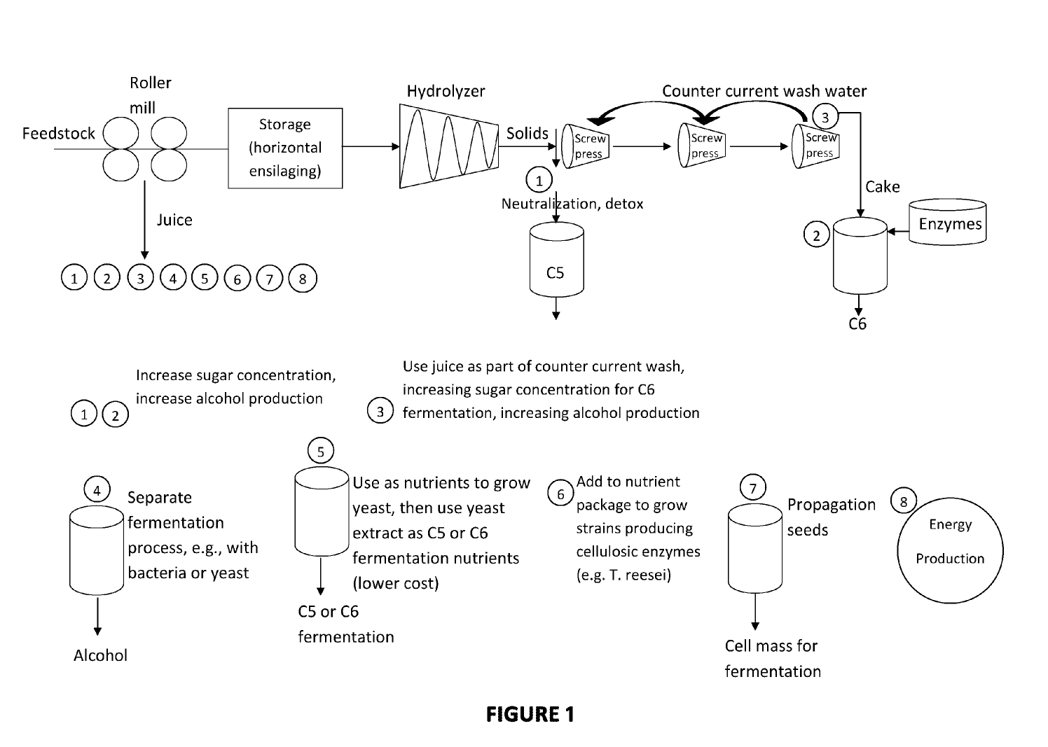

Specifically, the instant invention provides eight locations in the ethanol production process that juice can be reintroduced to increase the efficiency or yield of the ethanol production process. These eight locations are set forth schematically in FIG. 1.

There are benefits to removing the juice and the associated sugars during the milling process. For example, a dewatered feedstock allows for a lower liquid:solid ratio in the hydrolyzer, thereby requiring less energy and steam to bring the biomass up to temperature. Moreover, at a low liquid:solid ratio, the hydrolyzed sugars will be present in less water and, therefore, the sugar concentration will be higher, thus allowing for a higher ethanol concentration to be achieved during fermentation.

Moreover, allowing free sugar such as the sugar present in the juice to enter the hydrolyzer is a liability. In the hydrolyzer, sucrose is split to make glucose and fructose. In the hydrolyzer, the fructose is then transformed to 5-hydroxymethyl furfural (5-HMF).

This transformation to 5-HMF directly impacts the production of ethanol. The ethanol equivalents of the fructose are lost to the hydrolysis process. Moreover, 5-HMF is a potent fermentation inhibitor and its presence hinders fermentation. Additionally, high concentrations of juice in the feedstock have been observed to facilitate the build-up of excessive char/coke in the hydrolyzer.

When feedstock is roller-milled, the structure of the fibers is broken and the material is dewatered such that it can be stored for longer periods of time. Acid, which is used in the hydrolyzer, mixes better with the milled fiber and better permeates the feedstock during hydrolysis. If the feedstock were roller-milled, harvesting could proceed faster in the field because the feedstock would not have to be chopped as finely to facilitate acid diffusion and hydrolysis of cellulose and hemicellulose.

Removing a majority of the sugar will lower the fuel for microbes to heat the feedstock storage pile. This would allow for feedstock to be stored for a longer period of time after harvest.

If the sugars are not removed immediately after harvest, the sugar will be lost to microbes in the feedstock. Organic acids and other fermentation inhibitors are also likely by-products of microbial consumption of the cane juice sugars.

If the juice containing sugars is removed from the feedstock, there are several locations where the juice can be reintroduced in the process or used to facilitate higher ethanol conversion. The eight locations are numbered below as they are represented schematically in FIG. 1.
1. The juice could be added directly to the C5 sugar mixture after it has been isolated from the hydrolyzer in order to increase the sugar concentration in this mixture. This would result in an increased sugar concentration and ultimately an increased amount of ethanol produced in the C5 fermentation step.
2. The juice could be added directly to the C6 sugar mixture in order to increase the sugar concentration in this mixture. This would result in an increased sugar concentration and ultimately an increased amount of ethanol produced in the C6 fermentation step.
3. The juice could be used as the counter current wash water and the juice could be used to better wash the cake without lowering the C5 sugar concentration. Because certain microorganisms used to ferment C5 sugars, e.g., K. oxytoca, are sensitive to residual hydrolyzate, washing is desirable to ensure efficient fermentation.
4. The juice can be separately fermented once isolated from the milling process. The juice for this separate fermentation process could be concentrated to increase the efficiency of the process. This fermentation process could be carried out by yeast or bacteria.
5. The juice can be used to grow yeast to make yeast extract for use as nutrients for the C5 or C6 fermentation process.
6. The juice can be used as nutrients to grow various strains used to produce cellulosic enzymes, for example, T. reesei, used in the C6 fermentation process.
7. The juice can be used to grow strains that are used to grow yeast or bacteria used to ferment C5 and C6 sugars into ethanol.
8. The juice can be sent to an anaerobic digester, for example, in a waste water plant to make biogas that can be used for energy to run, for example, the roller mill or hydrolyzer.

If the juice were reintroduced into processes using either option 1 or option 2, sugar concentrations below 10% would not be detrimental.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Feedstock Processing and Sugar Analysis

Experiments were performed using fresh, forage-chopped energy cane to determine if a roller mill could be used to effectively dewater forage-chopped energy cane without significantly compromising particle size. As a side experiment, freshly cut energy cane was also roller-milled to determine the starting sugar concentrations in energy cane. For the cane being examined, the juice from the first press (primary juice) contained 134 g/L of reducing sugars (sucrose, glucose and fructose) equivalents and the aggregate juice mixed from multiple presses contained 104 g/L reducing sugars. The amount of juice present in the cane was calculated by subtracting the bound water (using derived values in the sugar cane industry) from the total moisture present in the energy cane. For the energy cane that was used in the experiments and calculations, the percent total reduce sugar (TRS) for the wet material was 6.7%. The concentration of the sugar in the juice was measured via HPLC.

If a juice-extraction process were used to recover the sugar in the energy cane in a bio-refinery, the following amounts of ethanol could be expected. The following calculations are exemplary and makes assumptions about size and amount of feedstock being used. As one of skill in the art will understand, these calculations show that the increase in ethanol production is significant.

For the following calculations, the numbers are based on a 36 million gallon per year production plant. Assuming a concentration of reducing sugars at 10% (˜100 g/L) in the juice and sugar refinery yields, this corresponds to an increased ethanol yield of 28 gallons/OD ton of feedstock. This amounts to an extra 26,900 gallons of ethanol/day and an extra 8.8 million gallons of ethanol/year.

If the concentration of reducing sugars is estimated at only 5% (˜50 g/L) in the juice and sugar refinery yields, this corresponds to an increased ethanol yield of 14 gallons/OD ton of feedstock, or an extra 13,400 gallons of ethanol/day and an extra 4.4 million gallons of ethanol/year. Tables showing the increase in efficiency of the process when using the juice are set forth as Tables 1-7 below.

TABLE 1

| | Wet Fresh | | OD Weight |
|---|---|---|---|
| | Eenegy Cane | 10% OD EC | Composition |
| Total Reducing | 6.7% | 24.0% | 0% |
| Sugar | | | |
| Cellulose | 9.2% | 33.0% | 45% |
| Hemicellulose | 4.8% | 17.2% | 23% |
| Lignin | 5.8% | 20.8% | 27% |
| Ash | 1.4% | 5.0% | 5% |
| Moisture | 72.0% | 0.0% | 0% |
| Totals | 100.0% | 100.0% | 100.4% |

TABLE 2
10% Sugar in Juice

| | Fresh EC | | OD | |
|---|---|---|---|---|
| TRS | 6.7% | Fiber | | |
| Cellulose | 9.2% | (C, HC, L) | | |
| Hemicellulose | 4.8% | 19.9% | | |
| Lignin | 5.8% | | | |
| Ash | 1.4% | Bound H₂0 | Juice | Sugar % |
| Moisture | 72.0% | 5.36% | 66.6% | 10.1% |
| Totals | 100.0% | | | |

TABLE 3

| | OD Weight |
|---|---|
| 5% OD EC | Composition |
| 12.0% | 0% |
| 38.6% | 45% |
| 20.1% | 23% |
| 24.3% | 28% |
| 5.0% | 5% |
| 0.0% | 0% |
| 100.0% | 100.3% |

TABLE 4
5% Sugar in Juice

| | Fresh EC | | | |
|---|---|---|---|---|
| TRS | 3.4% | Fiber | | |
| Cellulose | 10.8% | (C, HC, L) | | |
| Hemicellulose | 5.6% | 23.2% | | |
| Lignin | 6.8% | | | |
| Ash | 1.4% | Bound H₂0 | Juice | Sugar % |
| Moisture | 72.0% | 6.26% | 65.7% | 5.1% |
| Totals | 100.0% | | | |

TABLE 5

| | | OD Metric tons/Day | |
|---|---|---|---|
| | | 10% TRS | 5% TRS |
| CP1 Run Rate = 40 OD mT/hr | | 230.4 | 114.9 |
| 85% | Extraction | 195.84 | 97.63 |
| 94% | Clarification | 184.09 | 91.77 |
| 90% | Fermentation | 84.50 | 42.12 |
| 98% | Distillation | 82.81 | 41.28 |
| 97% | Dehydration | 80.32 | 40.04 |
| 0.789 kg/L | L EtOH/Day | 101,804 | 50,750 |
| 3.78 L/gal | Gal EtOH/Day | 26932 | 13426 |
| | Gal EtOH/Year | 8,887,608 | 4,430,578 |
| | (330 day) | | |
| | Extra Gallons/OD ton | 28.05 | 13.99 |

TABLE 6

| | Brix | Furfural | Sugars | % | % |
|---|---|---|---|---|---|
| Sample | (%) | (g/L) | (% w/v) | Solids | Insolubles |
| CPM | 15.3 | | 13.46 | | |
| #2629.1 | | | | | |
| CPM | 12.5 | | 10.42 | | |
| #2629.3 | | | | | |

TABLE 7

| Suc- | | | | Carbo- | | | |
|---|---|---|---|---|---|---|---|
| cinic | Lactic | Formic | Acetic | hydrate | | | |
| Acid | Acid | Acid | Acid | Break- | | Cello | |
| (g/L) | (g/L) | (g/L) | (g/L) | down | Sample | (g/L) | Glu (g/L) |
| 0.832 | 1.269 | 6.684 | ND | | CPM | ND | 34.846 |
| | | | | | #2629.1 | | |
| 0.677 | 1.554 | 6.046 | ND | | CPM | ND | 28.544 |
| | | | | | #2629.3 | | |

| | | | | | | Total |
|---|---|---|---|---|---|---|
| | | Ara | Man | Suc | Fru | Carbohydrates |
| Xyl (g/L) | Gal (g/L) | (g/L) | (g/L) | (g/L) | (g/L) | (g/L) |
| ND | ND | ND | ND | 68.540 | 31.239 | 134.63 |
| ND | ND | ND | ND | 49.942 | 25.689 | 104.18 |

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for preparing feedstock comprising:
milling the feedstock;
separating the juice and the bagasse; and
thereby preparing the feedstock.

2. The method of claim 1, further comprising allowing microbes to consume the residual soluble sugar in the bagasse after separating the juice.

3. The method of claim 2, wherein the microbes are present on the feedstock at harvest.

4. The method of claim 2, wherein the feedstock is inoculated with the microbes after the juice is extracted.

5. The method of claim 1, wherein the microbes are allowed to consume the residual sugar for 1 day, 2 days, 3 days, 4 days, 5 days, 6 days, 7 days, 8 days, 9 days, 10 days, 11 days, 12 days, 13 days or 14 days.

6. The method of claim 1, wherein the prepared feedstock is fermented to produce ethanol.

7. The method of claim 6, wherein the juice is added back to the bagasse mixture prior to fermentation.

8. The method of claim 4, wherein the bagasse is hydrolyzed prior to addition of the juice.

9. A method of fermenting processed feedstock as depicted in FIG. 1.

10. A method of increasing the amount of ethanol produced from feedstock by processing the feedstock as depicted in FIG. 1.

11. The method of claim 1, wherein the feedstock is sugarcane.

12. A method of increasing the efficiency of ethanol production by fermentation of processed feedstock by a microorganism comprising:
milling the feedstock and collecting the juice, wherein the juice comprises sugar,
using the juice during the feedstock preparation or fermentation; and
thereby increasing the efficiency of ethanol production.

13. The method of claim 12, wherein the efficiency is increased as compared to a method in which the juice is not used.

14. The method of claim 12, wherein the juice is added to the fermentation broth.

15. The method of claim 14, wherein the fermentation broth is C5 sugar fermentation broth.

16. The method of claim 14, wherein the fermentation broth is C6 sugar fermentation broth.

17. The method of claim 12, wherein the juice is used as a wash for a liquid/solid separation.

18. The method of claim 12, wherein the juice is fermented separately from the bagasse extract.

19. The method of claim 12, wherein the juice is used to grow yeast to produce yeast extract.

20. The method of claim 19, wherein the yeast is used to produce yeast extract.

21. The method of claim 12, wherein the juice is used to grow microorganisms that produce cellulosic enzymes.

22. The method of claim 21, wherein the cellulosic enzymes are used in the preparation of C6 sugar extract.

23. The method of claim 12, wherein the juice is used to culture bacteria or yeast for fermentation of C5 or C6 sugar extract.

24. The method of claim 12, wherein the feedstock is energy cane.

25. The method of claim 24, wherein the energy cane is sugar cane.
